(19) United States
(12) Patent Application Publication
(10) Pub. No.: US 2003/0104617 A1
(43) Pub. Date: Jun. 5, 2003

(54) Layer vessel matrix for universal tissue building and device for incubation

(76) Inventor: Matthias Schreiber, Erfurt (DE)

Correspondence Address: COLLARD & ROE, P.C., 1077 Northern Boulevard, Roslyn, NY, 11576, US

(21) Appl. No.: 10/309,604

(22) Filed: Dec. 4, 2002

(30) Foreign Application Priority Data
Dec. 4, 2001 (DE) 101 59 426.7

(51) Int. Cl. C12N 5/08; B28B 11/08; B29C 49/08; B29C 55/00

(52) U.S. Cl. 435/372; 264/292

(57) ABSTRACT

A procedure and a device for the production of vascular systems, with cell cultures being applied to support structures, uses a liquid to perform a pressing. The liquid used is a nutrient medium adjusted to the cell culture at a tuned atmosphere; a culture support membrane is put on a negative mould; press needles are pressed into this layer. Subsequently, a preliminary pressing is performed using a positive mould; after this, two negative moulds with corresponding positive mould are applied and the positive moulds are removed. The press needles are removed from one mould; both negative moulds are joined to one another. The press needles are arrested in the mould; press core needles located within the press needles are removed. A circulation pump system is connected to the press and flush needles and liquid is pumped through these.

### BACKGROUND OF THE INVENTION

1. Field of the Invention

The present invention relates to a procedure for the production of a vessel matrix and to a device for incubation, particularly for incubation in the human body. The invention provides for the application of cell cultures to support structures and for their shaping in a press and for their culturing procedure. The device for incubation comprises nesting tissues and their supply vessels.

2. The Prior Art

The use of a culture matrix for building single blood vessels and their implantation without essential rejection symptoms is well known.

But vessels prepared like this are only applicable with a major surgical effort as tissue nesters (culture and vessel matrix).

A particular drawback of this is the lack of supporting and connective tissue as well as a high mechanical susceptibility of the cell cultures thereon because of lacking capillary structuring.

### SUMMARY OF THE INVENTION

It is an object of the present invention to provide the anatomically correct building of a large-area vascular system or single vessel for the creation of a frame and supply matrix for a culture of large-area tissue units at a low cost.

The above object is achieved in accordance with the invention by providing a procedure for the production of a vascular system, with cell culture being applied to support structures, and with a liquid being used to perform a pressing, comprising the steps of

adjusting the liquid used as a nutrient medium to the cell culture at a tuned atmosphere;

putting a culture support membrane layer on a negative mould;

pressing press needles into this layer;

subsequently, performing a preliminary pressing using a positive mould;

after this applying two negative moulds with corresponding positive mould and removing the positive mould;

removing the press needles from one mould;

joining both negative moulds to one another;

arresting the press needles in the mould;

removing press core needles located within the press needles; and

connecting a circulation pump system to the press and flush needles and pumping a liquid there through.

The present invention also provides a device for incubation, in the human body, in which nesting tissue and its supply vessels are arranged, comprising an access unit and a nest unit, wherein

the access unit contains an abdominal passage tube 1.4, at which are arranged an outer growing-in area 1.3 and an inner growing-in area 1.3.1 as well as an abdominal plug 1, while the abdominal plug 1 is fastened by a holding and sealing ring 1.1, and allows access to the flushing channel 4.1 and to the flushing-in channel 5;

the nest unit contains at least one nest ground vessel 6 with two supply vessels 6.3, while the supply vessels 6.3 run through an abdominal access hose 2 into the nest area and a vessel inlet and outlet opening 3.1 allows the unpressurised inlet or, correspondingly, outlet of the supply vessels 6.3; and

an egg flushing-in channel 5 leads from the outside to the nest ground vessel 6.

The invention provides for the supply to different cells and cell types. These can be obtained from a cell culture without requiring the use of embryonic stem cells for such culture. It rather provides for the supply to any cell. This makes it possible to use this way to imitate various organs as, e.g., uterus, bladder, kidneys, and liver as well as tissue, while fluids created by their metabolic functions need to be drained off for the latter two, using a separate fluid basic system.

The example explains the preparation of the nest ground vessels for building a uterus sample tissue in a combined press and culture procedure.

Different culture support membranes (filter membranes) are available for pressing. Biodegradable synthetic materials, proteins like hens' eggs and catgut materials or membranes consisting of bacterial cellulose as well as other appropriate culture supports are used for this purpose. It is also possible to use semi permeable membranes as a protective shield against the immunological system (e.g., synthetic membrane), for which additional materials can be applied by coating or pasting according to the corresponding application.

The cell cultures used for creating the vascular matrix are aceterbacterxylinum or used strains, cellulose formers or direct culture mammal cells which are applied to the culture support membrane before pressing. The culture coats used for single supports are, e.g., vascular endothelia, non-striated muscles and supporting tissue, while the additional coating used is tunica adventitia, media, or interna.

A first example explains the building of the vascular cell culture.

Herein, pressing is performed by means of a liquid, which fixes premoulded structures in their positions and is used as a nutrient medium for the cell cultures put on. The pressing liquid used is a nutrient medium for mammal cells.

A typical medium for the culture of mammal cells is composed as follows.

| Amino acids | Vitamins | Salt | Others |
|---|---|---|---|
| Arginine | Biotin | NaCl | Glucose |
| Cysteine | Choline | KCl | Penicillin |
| Glutamine | Folate | NaH₂PO₄ | Streptomycin |
| Histidine | Nieotinamide | NaHCO₃ | Phenol red |
| Isoleucine | Pantothenate | | Complete serum |
| Leucine | Pyridoxal | CaCl₂ | |
| Lysin | Thiamine | MgCl₂ | |
| Methionine | Riboflavine | | |
| Phenylamine | | | |
| Threonine | | | |
| Tryptophane | | | |
| Tyrosine | | | |
| Valine | | | |

Glucose is used in a concentration of 5 to 10 mM.

The amino acids are all present in the L form, and are used with one or two exceptions in concentrations of 0.1 or 0.2 mM, while vitamins are used in a 100 times smaller quantity with approx. 1 μM.

The Serum is normally obtained from horses or calves. It amounts to up to 10% of the total volume.

Penicillin and streptomycin are antibiotics which shall suppress bacterial growth. Phenol red is used as a pH indicator, the colour of which is checked to maintain a pH of approximately 7.4 at 37° C. in an atmosphere of 5% CO₂/95% air.

The nutrient media, which may be used, include L15 or DMEN or DMEN 1 to 1 MEM-Earl or RPM/1640 or NCTC-135, F 12 as well as Mc Coys 5 a or an endothelium medium tuned to the cells.

Costem formulation medium+ECGS+1% penicillin/streptomycin solution and blood substitute substances.

The development of the cell culture is done at temperatures of about 37° C., approximately, and in an atmosphere tuned to the culture. To enable growth in the various embodiments, and to control the growth process, the nutrient medium can be modified in its nutritive values, thus tuning it to the corresponding conditions of the culture. A membrane is placed without creases on a negative mould. The biomembrane is moist and provided with a culture coating supporting tissue. After this, the press needles are put on and pressed in, and, subsequently, a positive mould is used to press the big vessels. After removing the positive mould and the press needles, arteries and veins are coated with fibronectin or other factors. A membrane with a culture coating from non-striated muscle cells is placed on a separate mould. This is a positive mould for which pressing is done using a negative mould. This mould is removed after the desired forming to shape of the membrane.

Projecting material is removed from the structures created using a punching process. They remain on the positive mould. The non-striated muscle cell layers needed for the lining of arteries and veins are introduced into the structure by placing onto the negative mould. The positive mould is removed, the structures are formed to be shaped and remain in the negative mould. This operation is repeated until arteries and veins are lined with a membrane with non-striated muscle cells. After this, coating is performed using fibrenectin or other appropriate means. A membrane with a culture coating vascular endothelium is placed on a separate mould. This is also a positive mould for which a pressing is done using a negative mould. This mould is removed after the desired forming and shaping of the membrane.

A punching process is used to remove material not needed from the structures being created, and they are applied with the positive mould to the negative mould and fixed. Furthermore, it is possible to introduce endothelium-coated single bodies, e.g. venous valves. According to the embodiments, the membranes have a total layer thickness of between 40 and 300 μm which determines the distance between the compression moulds. To avoid damage to the culture during pressing, the distance between the membrane and the compression mould should be set to between 0 and ±0.3 μm.

After this, the press needles are put on, they should be adjusted with their distance to the compression mould such that they are irrigated by the nutrient medium during the entire pressing and culture phase. Then, the second negative mould, which has been treated in the same way, is taken and they are assembled. The press needles are arrested.

Now, it is possible to prepare the intermediate connective layers, which form the adhesion area of the culture, for the culture by glueing, using collagen type 1 or adhesion factors poly-P-lysine or fibronectin. This can also be done by other means if necessary.

Both negative moulds are put up, assembled, and joined to one another. After this, the flexible tubes are connected to the compression mould, and, during the next 15 minutes, the big distribution vessels are selected individually for pressing, thus bringing about the forming and shaping of the small vessels.

A nutrient medium suitable for the cell culture is used for this purpose. A circulatory system is connected after removing the press needles. Depending on the size, a liquid volume of 50 to 1,000 ml is pumped through the mould in one minute. The nutrient medium used for this purpose is also used as a growth indicator. It can determine cell growth through the consumption of nutrients (colour change). Culture time is over when the intermediate connective layers have grown together, i.e., that the connective layer width of 0.3 mm is closed completely. Due to different cell masses, layer thicknesses and reproductive capabilities, culture time differs widely and is limited to a range of 10 to 120 hours.

The vessels are removed from the mould after their maturation. Cleaning and pre-processing is done prior to utilization. Utilizations may include, e.g., the surgical building of desired structures, or the application of tissue cultures. It is also possible to use biohybrid systems, i.e., a combination with cladding membranes which protect the cell against attacks by the immunological system.

To avoid their occlusion, the vessels must always be filled with liquid. This is guaranteed by a circulatory system. The basic requirement to be met for a successful culture is clean work without causing any damage. Membranes both with and without cell application can be used for this procedure. To meet different objectives, they can be used either in combination or as single components.

Another example will explain the preparation of a nest ground tissue for a uterus with a combined press and culture procedure.

In this case, pressing is performed using a liquid. The pressing liquid used is a nutrient solution for Aceobacter xylinum according to Hestrin and Schramm, or other appropriate media. This liquid contains in weight percent:

2% glucose

0.5% peptone

0.5% yeast extract

0.27% NaH₂PO₄ and

0.16% citric acid (mononitrate),

and it is set using NaOH or HCl to a pH-value of 6, and to an oxygen content which is tuned to the cell culture.

For pressing, this liquid is diluted using 60% to 80% by weight of water, while it must still maintain a pH-value of 6 in addition to the tuned oxygen content.

A layer of membrane cellulose is put on the negative mould. The membrane is wet and moist, and still contains Aceobacter xylinum cultures. Depending on the embodiment, the membrane has a total layer thickness of 50 to 250 μm. After this, press needles are put on and pressed in and, subsequently, preliminary pressing is performed using a positive mould. After approximately 10 minutes, two negative moulds are taken again with the corresponding positive mould, and the positive mould, which has brought about a preliminary pressing of the membrane, is removed. Subsequently, the press needles are removed from one mould. In the mould which still keeps the press needles, the areas, which cannot be reached by the pressing liquid to a sufficient extent during later pressing, are thoroughly wetted using an initial nutrient solution according to Hestrin and Schramm. Both negative moulds are put up, and assembled, and joined to one another. The press needles are arrested in the mould.

After this, the flexible tubes are connected to the press core needles, and, during the next 30 minutes, the big distribution vessels are selected individually for the pressing, and injected. A nutrient solution according to Hestrin and Schramm, which presents a dilution to 60 to 80% of the initial solution, is used for pressing. Pressing is done at a pressure of 3 bars.

Pressing is complete after 30 minutes and 4 modulations for the pressing of every main distribution vessel. The press core needles are removed, and a circulation pump system is connected to the press and flush needles. The covering at the compression mould is removed, such that the pressing liquid drain is released, and these areas can be reached by the nutrient liquid.

A liquid volume of one liter is pumped through this mould in one minute. The liquid used for this is a 20% to 40% by weight nutrient solution according to Hestrin and Schramm with a pH-value of 6. It has a temperature of 24° C. to 30° C., and it is mixed with a culture of Aceobacter xylinum. This presents a culture density of 2.6×10 to 3.2×10. At a grown layer thickness of 50 to 250 μm after a culture time of 6 to 48 hours, the nutrient medium is flushed until the vessels are clean. After this, they are washed using a medical soap at 90° C. for 30 minutes. The cleaned vessels are provided with a culture coating of vascular cells.

Corresponding punctures must be made for further preparation as the membrane of the nest ground vessels consisting of cellulose only has a pore size of 0.1 μm, and thus does not allow tissue insertion and the supply to the nest cells. For this purpose, the membranes of the basic vessels must be perforated using laser or other means, such that the perforations present a sufficient size which allows the formation of capillary vessels to a sufficient extent. The function of this is to allow that corresponding capillary vessels can form at the perforation points and supply the corresponding nest cells. A perforation key of 6 to 1 is required, i.e., 6 perforations are made for every capillary vessel in order to obtain sufficient opportunities for access to the blood stream.

After perforating the membrane layer, it is necessary to close the perforation points by vascular cells. This allows the creation of a closed vascular system for implantation, such that no haemorrhagic risk is created by the perforation. For this purpose, the membranes must be provided with a coat of vascular cells. Different structures and forms can be developed by analogy with this procedure, while prefabricated structures may be introduced as well, and will grow together in the corresponding culture procedures.

The perforation of all systems formed according to this variant is necessary to comply with the functional tasks. To ensure the sufficient drainage of the liquids formed by the cell cultures, it is necessary to keep in mind that bigger perforations are required for liquid systems, and that the capillary vessels required for the cell cultures can only develop to a sufficient extent under medicamentous control. Furthermore, it may become necessary under certain circumstances to support the cell cultures using a drug which promotes oxygen supply in order to achieve a sufficient maturation of the cell cultures. Even further, it is also possible to achieve the maturation of the vascular system and of the nest culture outside the body with a corresponding supply so as to keep patient stress low during implantation. This may be useful in particular for implants with a metabolic function as metabolites may cause considerable damage to these cell cultures, such that a sufficient function would not be provided any longer.

### BRIEF DESCRIPTION OF THE DRAWINGS

Other objects and features of the present invention will become apparent from the following detailed description considered in connection with the accompanying drawing which discloses several embodiments of the present invention. It should be understood, however, that the drawing is designed for the purpose of illustration only and not as a definition of the limits of the invention.

In the accompanying drawings:

FIG. 1 is a section through the entire device;

FIG. 2 is a section through the abdominal plug;

FIG. 3 is a view showing the arrangement of the egg flushing-in channel;

FIG. 4 is a view of the vessel access;

FIG. 5 is a view of the flushing channel;

FIG. 6 is a section through a nest ground vessel; and

FIG. 7 is a view of a press mould.

The device according to the invention represents a universal incubator, and comprises an access unit, a control unit, and a nest unit.

### DETAILED DESCRIPTION OF PREFERRED EMBODIMENTS

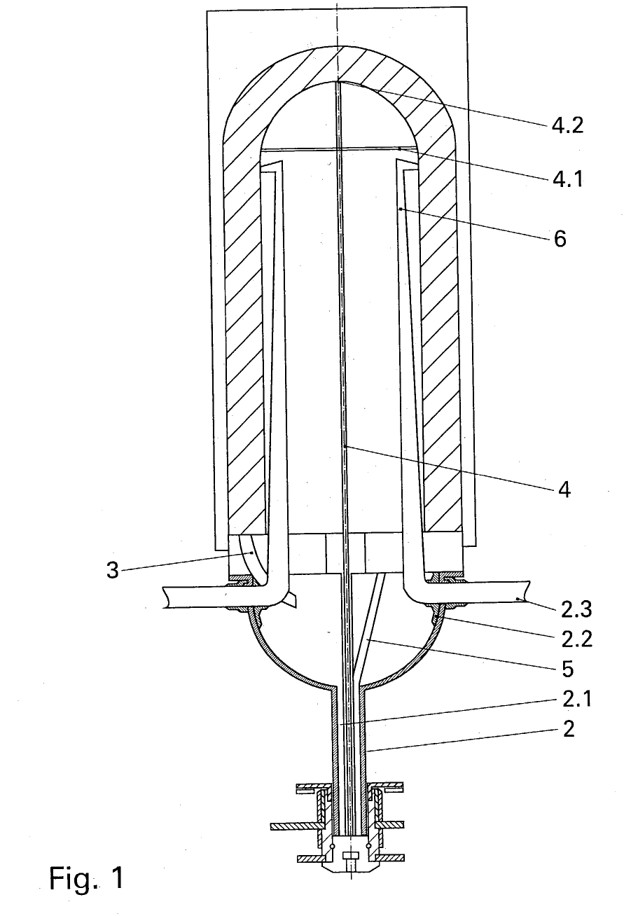

FIG. 1 shows the general arrangement. The abdominal access hose 2 divides the implant from the abdominal space. It provides access to the nest space, and constitutes the drainage channel for the flushing liquid and for the flushed material from the nest area.

The fastening position of the abdominal access hose 2 at the abdominal passage tube 1.4 is the adhered surface 1.9 as shown in FIG. 2.

The protective gel 2.1 forms the outside termination of the abdominal access hose. It closes the latter outside of the flushing times, thus forming a germ barrier.

The inner vessel sleeve 2.2 provides a mechanical protection for the two supply vessels which run through the abdominal access hose 2 into the nest area. This consists of bacterial cellulose while tissue grows through and clads it, thus presenting another germ barrier.

The outer vessel sleeve 2.3 forms a mechanical protection for the two supply vessels which run through the abdominal access hose 2 into the nest area. These consist of bacterial cellulose while tissue grows through and clads them, thus presenting another germ barrier.

The compression liquid valve 3 provides access to the compression liquid and thus ensures compression control and the removal of the compression liquid before germ withdrawal, such that penetration into the abdominal space is prevented.

The flushing channel 4 allows the external access to the nesting tissue and ensures the function during application as a uterus, thus enabling periodic access to the nest ground. It ensures the cleaning of the nest area using the flushing nozzles 4.1 and the flushing head nozzle 4.2.

The flushing nozzles 4.1 allow the flushing of the nest ground to clean the flushing material which occurs periodically.

The flushing head nozzle 4.2 allows the flushing clean of the head of the nest tree, and the controlled distribution of the flushing material (nest tree ball catheter).

In this embodiment, the egg flushing-in channel 5 allows the flushing-in of the fertilised ovocyte, and thus also the positioned nesting in the nest ground which is located on the nest ground vessels 6.

The access unit shown in FIG. 2 contains the abdominal passage tube 1.4 in which an abdominal plug 1 is used as a detachable shutter. The abdominal plug 1 is both a mechanical and a germ barrier, and provides access to the flushing channel 4 and to the egg flushing-in channel 5, and thus to the nesting tissue and its control.

A holding and sealing ring 1.1 secures the abdominal plug 1 in the abdominal passage tube 1.4. It prevents an exchange of gases or fluids so as to avoid a conveyance of germs into the implant or, correspondingly, into the abdominal space.

The protective ring 1.2 has the function of protecting the border of the wound around the implant against mechanical influences. The protective ring 1.2 is fastened securely to the abdominal passage tube 1.4, and thus allows a traction relief or pressure relief when placing or removing the abdominal plug 1. Furthermore, the protective ring 1.2 is used to cover wound or healing ointments which are applied to the border of the wound.

The outer growing-in area 1.3 and the inner growing-in area 1.3.1 present a metallic porous texture. It allows the application of a tissue insert, and thus the growing in of the implant.

The abdominal passage tube 1.4 is the access to the abdominal cavity or, correspondingly, to the implant, and it is also used as a germ barrier, thus avoiding infections of the abdominal space as these are sealed by the abdominal plug 1.

The tissue anchor 1.5 is used to form a tissue coat of the abdominal passage tube 1.4, and thus prevents the excessive loading of the wound borders of the implant. The tissue anchor 1.5 has a good grip porous surface so as to allow tissue insertion and its growing together with the abdominal wall. The holding tube 1.6 has the function of securing the tissue anchor 1.5 on the abdominal passage tube 1.4. The ring 1.7 consisting of bacterial cellulose prevents disintegrating cicatrisation and hernia formation by the connection with the punctured tissue. The abdominal termination ring 1.8 has the function of preventing the chafing of tissue or, correspondingly, intestine.

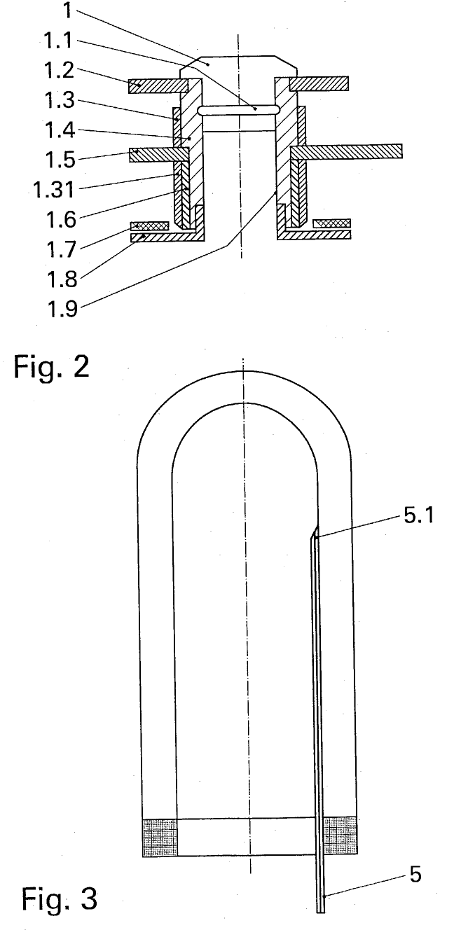

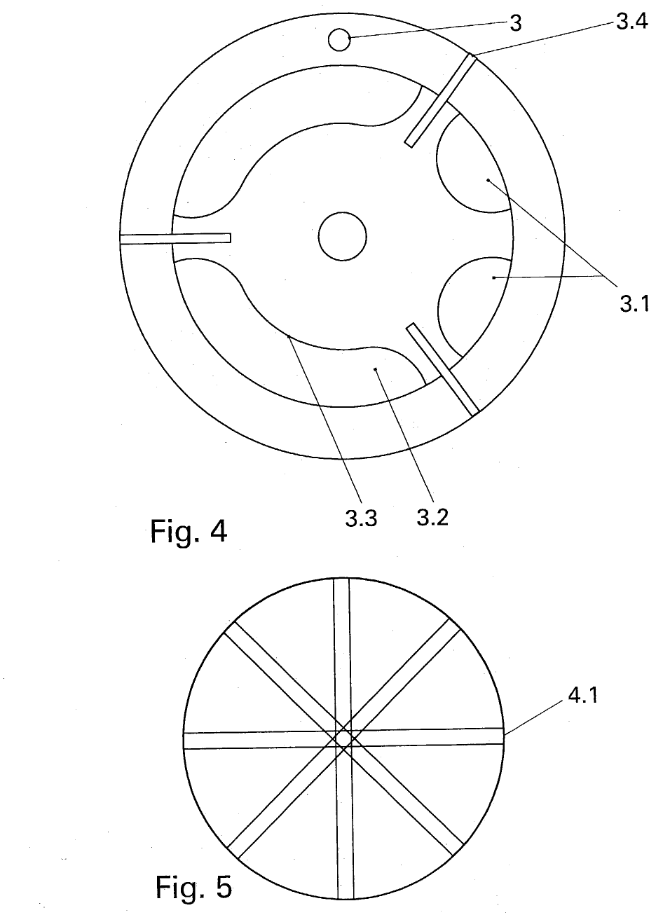

FIG. 3 shows the arrangement of the egg flushing-in channel 5 at the nest ground vessel 6. Through the egg flushing-in channel 5, the fertilised ovocyte is flushed in and nested in position in the nest ground. The egg flushing-in channel holder 5.1 secures the egg flushing-in channel via the nest ground at a place which is favourable for nesting.

FIG. 4 illustrates the vessel inlet and outlet. The vessel inlet and outlet opening 3.1 allows an unpressurised inlet or, correspondingly, outlet of the supply vessels 6.3. The access openings 3.2 ensure access to the germinal and nesting tissue as well as the smooth drainage of the flushing liquid and of the flushing material, thus allowing the cleaning of the nest space. When using the device as a uterus, a gel is used as a germ barrier after the completion of the 3rd month. The gel is applied at the point of support 3.3. Holding nails 3.4 are used to secure the gel plug in the stabilization ring.

FIG. 5 shows the distribution of the flushing liquid.

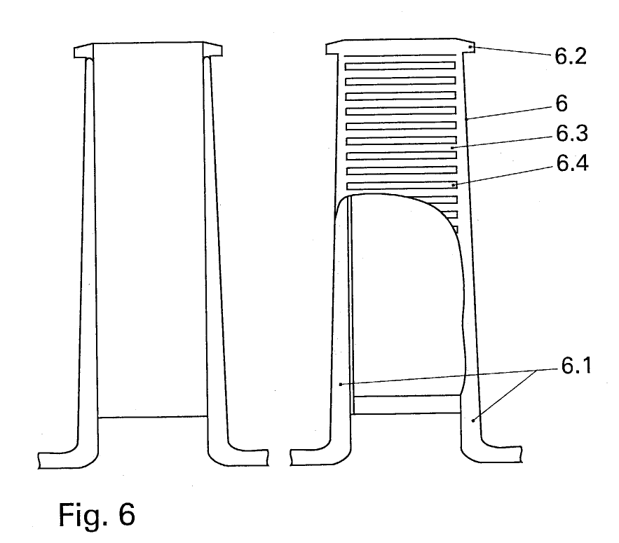

FIG. 6 shows the embodiment of the nest ground vessels 6. The nest ground vessels 6 comprise the supply vessels 6.3 of the nest ground or, correspondingly, the cell cultures deposited. The pore size of 0.1 μm requires the perforation by a laser of the nest ground vessels consisting of cellulose in order to allow the formation of access vessels. This creates openings with the size of capillary vessels which can grow in by themselves for the supply of the nest tissue. The main supply vessels 6.1 are formed by the inlet artery and by the exit vein. The holding and protection edge 6.2 holds and secures the nest ground vessels 6 on the nest tree. The supply vessels 6.3 form the suppliers belonging to the nest ground and formed completely to shape except for capillary vessels. The intermediate and connective layer 6.4 forms the boundary between the supply vessels and assumes the function of connective tissue. The nest edge 6.5 secures the nest ground vessels in their mould.

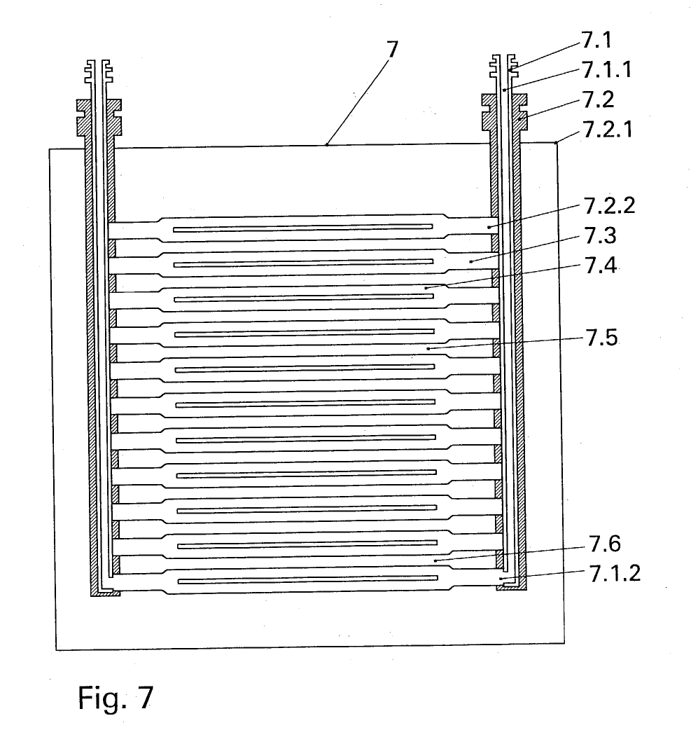

FIG. 7 shows the design of the compression mould 7. It consists of 3 parts, of the corresponding negative moulds which determine the outside of the nest ground vessels, and of the positive mould which allows the premoulding of the vessels.

The press core needle 7.1 has a press core needle channel 7.1.1 which is provided with an opening at its bottom end. The press core needle channel 7.1.1 allows the through conduction of the pressing liquid. The opening 7.1.2 of the press core needle channel 7.1.1 allows the controlled delivery of the pressing liquid, such that the distribution vessels 7.3 and the nest ground vessels 7.4 can be formed to shape. The press needle 7.2 allows the execution of the pressing and the corresponding after culture. It is provided with the flushing channel of the press needle 7.2.1, while this channel also forms the guide for the press core needle 7.1 and is provided with flush openings for the distribution ground vessels.

The flushing channel 7.2.1 of the press needle allows the flowing-through of the pressing or, correspondingly, nutrient liquid, such that the opening for the distribution vessels 7.2.2 can be selected. The opening 7.2.2 for the distribution vessels form the corresponding individual selection of the distribution vessels 7.3. The distribution vessels 7.3 form the corresponding single access of the nest ground vessels 7.4 connected to that. The nest ground vessels 7.4 provide the nest cultures with oxygen and nutrient. The intermediate connective layer 7.5 delimits the vessels, which have been pressed, and formed to shape by the after culture, among themselves, and forms the connective and supporting structure of the implant. Thus, it replaces the endogenous connective tissue. It secures the distribution vessels 7.3 and the nest ground vessels 7.4 in their positions, and thus provides a sufficient protection against mechanical influences. These flat and superimposed surfaces are also the main surfaces for the membranes to grow together.

The function of the pressing liquid drain 7.6 is to allow the drainage of the pressing liquid in certain areas of the compression mould, such that these areas can be reached by the nutrient liquid. This is only done for cellulose cultures before the intermediate area layers have grown together. This ensures that the membranes or, correspondingly, corresponding bacterial cultures can grow together. The pressing liquid drain 7.6 is closed by the compression mould covering 7.7 during pressing, such that the mould pressure remains constant. After pressing in the culture phase, the compression mould covering 7.7 is removed.

Accordingly, while a few embodiments of the present invention have been shown and described, it is to be understood that many changes and modifications may be made thereunto without departing from the spirit and scope of the invention as defined in the appended claims.

### LIST OF REFERENCE NUMERALS

1 Abdominal plug

1.1 Holding and sealing ring

1.2 Protective ring

1.3 Outer growing-in area

1.3.1 Inner growing-in area

1.4 Abdominal passage tube

1.5 Tissue anchor

1.6 Holding tube

1.7 Ring consisting of bacterial cellulose

1.8 Abdominal termination ring

1.9 Adhered surface

2 Abdominal access hose

2.1 Protective gel

2.2 Inner vessel sleeve

2.3 Outer vessel sleeve

3 Compression liquid valve

3.1 Vessel inlet and outlet opening

3.2 Access opening

3.3 Point of support for gel

3.4 Holding nail

4 Flushing channel

4.1 Flushing nozzle

4.2 Flushing head nozzle

5 Egg flushing-in channel

5.1 Egg flushing-in channel holder

6 Nest ground vessel

6.1 Main supply vessel

6.2 Holding and protection edge

6.3 Supply vessel

6.4 Intermediate and connective layer

6.5 Nest edge

7 Compression mould

7.1 Press core needle

7.1.1 Press core needle channel

7.1.2 Opening of the press core needle channel

7.2 Press needle

7.2.1 Flushing channel of the press needle

7.2.2 Opening for the distribution vessels

7.3 Distribution vessels

7.4 Nest ground vessels

7.5 Intermediate connective layer

7.6 Pressing liquid drain

7.7 Compression mould covering.

What is claimed:

1. Procedure for the production of a vascular system, with cell culture being applied to support structures, and with a liquid being used to perform a pressing, comprising the steps of
adjusting the liquid used as a nutrient medium to the cell culture at a tuned atmosphere;
putting a culture support membrane layer on a negative mould;
pressing press needles into this layer;
subsequently, performing a preliminary pressing using a positive mould;
after this, applying two negative moulds with corresponding positive mould and removing the positive mould;
removing the press needles from one mould;
joining both negative moulds to one another;
arresting the press needles in the mould;
removing press core needles located within the press needles; and
connecting a circulation pump system to the press and flush needles and pumping a liquid there through.

2. Procedure as claimed in claim 1, comprising
perforating membranes of the vessels and, after this, closing perforation points with vascular cells.

3. Device for incubation, in the human body, in which nesting tissue and its supply vessels are arranged, comprising an access unit and a nest unit, wherein
the access unit contains an abdominal passage tube 1.4, at which are arranged an outer growing-in area 1.3 and an inner growing-in area 1.3.1 as well as an abdominal plug 1, while the abdominal plug 1 is fastened by a holding and sealing ring 1.1, and allows access to the flushing channel 4.1 and to the flushing-in channel 5;
the nest unit contains at least one nest ground vessel 6 with two supply vessels 6.3, while the supply vessels 6.3 run through an abdominal access hose 2 into the nest area and a vessel inlet and outlet opening 3.1 allows the unpressurised inlet or, correspondingly, outlet of the supply vessels 6.3; and
an egg flushing-in channel 5 leads from the outside to the nest ground vessel 6.

4. Device as claimed in claim 3,
wherein at the abdominal passage tube 1.4, a flushing channel 4 is arranged which is connected to a flushing nozzle 4.1 and to a flushing head nozzle 4.2.

5. Device as claimed in claim 3,
wherein the abdominal passage tube 1.4 is connected to main supply vessels 6.1 which are formed by an inlet artery and an exit vein.

6. Device as claimed in claim 3,
wherein in the nest ground vessel 6, a compression mould 7 is arranged which comprises three parts, two negative moulds, which determine the outside of the nest ground vessels, and a positive mould which allows a premoulding of the vessels.

7. Device as claimed in claim 3,
wherein in the compression mould 7, press needles 7.2 are attached, which has a flushing channel 7.2.1 of the press needles 7.2, which also forms the guide for press core needles 7.1 and has flushing openings for the distribution ground vessels and that the press core needles 7.1 have press core needle channels 7.1.1 which are provided with an opening at their lower end.

8. Device as claimed in claim 3,
wherein a protective ring 1.2 is fastened solidly to the abdominal passage tube 1.4.

9. Device as claimed in claim 3,
wherein at the abdominal passage tube 1.4, a tissue anchor 1.5 is fastened which has a good grip porous surface and is connected to a holding tube 1.6.

10. Device as claimed in claim 3,
wherein an adhered surface 1.9 is attached as a fastening point of an abdominal access hose 2 at the abdominal passage tube 1.4.
